(19) United States
(12) Patent Application Publication
(10) Pub. No.: US 2010/0112101 A1
(43) Pub. Date: May 6, 2010

(54) BIO-AVAILABILITY/BIO-EFFICACY ENHANCING ACTIVITY OF STEVIA REBAUDIANA AND EXTRACTS AND FRACTIONS AND COMPOUNDS THEREOF

(75) Inventors: Ganga Raju Gokaraju, Andhra Pradesh (IN); Rama Raju Gokaraju, Andhra Pradesh (IN); Cletus D'souza, Karnataka (IN); Elizabeth Frank, Karnataka (IN)

Correspondence Address: KRAMER & AMADO, P.C., 1725 DUKE STREET, SUITE 240, ALEXANDRIA, VA, 22314, US

(73) Assignee: LAILA IMPEX, Vijayawada (IN)

(21) Appl. No.: 12/610,502

(22) Filed: Nov. 2, 2009

(30) Foreign Application Priority Data
May 6, 2008 (IN) 1115/CHE/2008

(51) Int. Cl. A61K 36/28; A61K 36/9066; A61P 19/02; A61P 9/12

(52) U.S. Cl. 424/737; 424/756; 424/769; 424/773; 424/774; 424/779

(57) ABSTRACT

The present invention discloses bio-efficacy and bio-availability enhancing composition comprising, an effective amount of stevia compound selected from raw powder or extract, or fraction(s) or compound(s) or combinations thereof derived from the whole plant or plant parts of genus Stevia, preferably leaves of Stevia rebaudiana for enhancing the bio-availability and bio-efficacy of therapeutic agents, cosmetic agents, supplements, food ingredients and beverages selected from but not limited to pharmaceutical drugs, nutrients (micro and macro nutrients), vitamins, minerals, Proteins, amino acids, enzymes, nutraceuticals, herbal drugs/products and antioxidants.

### FIELD OF INVENTION

The invention relates to novel bioenhancing agents comprising raw powder or extract, or fraction(s) or compound(s) derived from the leaf, stem, root, the whole plant of genus Stevia, preferably leaves of Stevia rebaudiana as a bio-availability facilitator and bio-efficacy enhancer. The invention also relates to compositions containing raw powder or extract, or fraction(s) or compound(s) derived from the leaf, stem, root, the whole plant or plant parts of genus Stevia, preferably leaves of Stevia rebaudiana as a bio-availability facilitator and bio-efficacy enhancer.

### BACKGROUND OF THE INVENTION

Improving the bio-availability of any drug candidate is the most important aspect of drug development. Bio-availability enhancement is extremely important and very often it is very essential for the successful outcome of drug candidates. Bio-availability enhancement can make the expensive drugs affordable and also very often reduce the toxic effects by reducing the required dose for many drug candidates. Modern drug development processes achieve oral bio-availability enhancement by a number of approaches, such as (a) increasing the polarity of the drug candidate through chemical modification, (b) salt preparation or complexation, (c) prodrug formation, (d) micronisation and nanonization, (e) specific polymorphic form selection, (f) targeted delivery of the drug to the site of action (g) controlled drug delivery through film coating (h) sustained drug release through polymorphic matrices formation (i) liposomal microencapsulation etc. However, enhancing the bio-availability through the supplementation of the main therapeutic agent with a secondary agent gained wide popularity since the traditional times.

Many plant products have been in use in popular traditional systems of medicine for hundreds of years as the bio-efficacy enhancing agents. A mixture of water extracts or herb powders derived from Piper nigrum, Piper longum and Ginger, popularly called as Trikatu, is widely used in traditional Ayurvedic medicine as an adjunct medicine for enhancing the efficacy of many traditional drugs. Piperine, a principle found in Piper nigrum and Piper longum has been prominently used with many nutraceutical agents and dietary supplements to enhance the bio-availability. The bio-availability of many pharmaceutical drugs was also found to be improved in the presence of Piperine. The standard dose of rifampicin, for example, could be significantly reduced by co-administering the drug with piperine as a bio-enhancing agent (Zutshi et al., “J Assoc Physicians India 33:223-224). Many natural agents have been known to possess potent bio-availability/bio-enhancing actions for a number of classes of drugs including antibiotics, antifungals, antivirals, anticancer, cardiovascular, anti-inflammatory/antiarthritic, antiTB, antileprosy, antihistaminic/respiratory, immuno-suppression and antiulcer.

Stevia, Stevia rebaudiana (BERT.) HEMSL. [Fam. Asteraceae], more commonly known as honey-leaf, has been used for centuries as a sweetener by the people of South America. The leaf extract and fractions have also been used for many years in the treatment and control of blood sugar among aboriginals in Paraguay and Brazil. The glycoside compounds present in stevia are mainly responsible for the sweetness. Stevia has the potential to be a natural non-calorific alternative to artificial sweeteners and sugar. Stevioside, the most abundant of the glycosides is over 200 times sweeter than sucrose. The other metabolites in stevia include austroinulin, rebaudiosides; dulcoside A; steviol; riboflavin; ascorbic acid; niacin and thiamin. Stevia is also a source of nutrients like calcium, magnesium, manganese, tin and zinc.

Stevioside and steviol found in the leaves of stevia is known to stimulate insulin secretion via a direct action on beta cells. Stevia reduces vascular tension and stevioside has been found to be an effective supplementary therapy for patients with hypertension. In countries like Canada and the United States, stevia leaf is available as over-the-counter herbal or dietary supplement product in health food stores. However, stevia and stevioside are not approved for sale as natural sweeteners in these countries. In contrast, stevia has been an approved natural sweetener in Japan and tons of stevia is consumed each year in many forms including processed foods like ice cream and beverages. Hot water extracts of stevia was found to inhibit the replication of human rotaviruses in vitro.

WO05048973A1 relates to an application of extract alone, and all it's active substances (individually), their microbiological and chemical modifiers and derivates, obtained from all varieties of Stevia rebaudiana cultivated from seed and nursery, in form that is suitable for oral usage leads to eliminating or reducing cellulite.

JP11269457A2 relates to a highly sweet component contained in leaves of Stevia rebaudiana BERTONI, a plant of the family Compositae, is extracted with water or hot water at 5-100° C. if necessary at a pH adjusted to about 10 with lime, etc., and is used as a sweetening agent. The extraction residue, i.e., the Stevia leaves discharged after the above extraction, is again subjected to extraction with a specified amt. of a hydrophilic org. solvent to give an antioxidant powder.

WO06116815A1 relates to a composition used to control cholesterol levels. Particularly, this document relates to a method for the treatment or prevention of disorders or diseases mediated by hyperlipidemia, which method comprises administering to a mammalian subject in need thereof an effective amount of a composition comprising an extract from at least one plant from the genus Stevia and at least one bile salt, salt thereof or derivative thereof admixed in a form suitable for therapeutic administration.

CN1328850A relates to a medicine for curing diabetes, in particular it is a Chinese medicine for curing diabetes. It is made up by using gynura segetun, dioscorea root, astragalus root, purslane, peach leaf as main materials, and using bittersquach, Stevia rebaudiana, alum, light calcium carbonate and water as auxiliary materials.

None of the prior art describes the bio-availability facilitating properties of the genus Stevia especially Stevia rebaudiana extracts or compounds or fractions or mixtures thereof.

### OBJECTS OF THE INVENTION

The main object of the present invention is to evaluate bio-efficacy and bio-availability enhancing actions of the agents comprising raw powder or extract or active fraction(s) or compound(s) derived from the leaf, stem, root or whole plant of the genus Stevia preferably the leaves of Stevia rebaudiana for enhancing the bio-availability of therapeutic agents, cosmetic agents, supplements, food ingredients and beverages selected from but not limited to pharmaceutical drugs, nutrients (micro and macro nutrients), vitamins, minerals, proteins, amino acids, enzymes, nutraceuticals, herbal drugs/products and antioxidants.

Still another object of the invention is to provide a bio-enhancer composition comprising raw powder or extract(s) or active fraction(s) or compound(s) derived from the whole plant or plant part(s) of the genus Stevia preferably Stevia rebaudiana leaf for enhancing the bio-availability of therapeutic agents, cosmetic agents, supplements, food ingredients and beverages selected from but not limited to pharmaceutical drugs, nutrients (micro and macro nutrients), vitamins, minerals, proteins, amino acids, enzymes, nutraceuticals, herbal drugs/products and antioxidants in the presence or absence of additives or excipients.

A further object of the invention is to provide a bio-enhancer composition comprising leaf powder or extract or active fraction or compound or compounds derived from Stevia rebaudiana leaf and optionally with known bio-enhancers such as piperine, Glycyrrhizin, Niaziridin, cumin extracts etc in combination with therapeutic agents, cosmetic agents, supplements, food ingredients and beverages selected from but not limited to drugs, nutrients (micro and macro nutrients), vitamins, minerals, proteins, amino acids, enzymes, nutraceuticals, herbal drugs/products and antioxidants.

Yet another objective of the present invention is to provide a process for isolating active fraction (s) from genus Stevia preferably Stevia rebaudiana.

Still another objective of the present invention is to provide a process for isolating non-bitter active fraction(s) from genus Stevia preferably Stevia rebaudiana useful as a bio-efficacy and bio-availability enhancing agent.

### SUMMARY OF THE INVENTION

The present invention discloses novel bioenhancing agents comprising raw powder or extract or active fraction(s) or compound(s) derived from genus Stevia preferably Stevia rebaudiana leaf, to potently enhance the bio-availability and bio-efficacy of a number of classes of drugs but not limited to antibiotic, antiobese, antidiabetic, antifungal, antiviral, anticancer, cardiovascular, anti-inflammatory, antiarthritic, antiTB/antileprosy, antihistaminic/respiratory, immuno-suppression, immune modulators, cholesterol lowering drugs, C-reactive protein inhibitors and antiulcer drugs.

The present invention further discloses novel bioenhancing compositions comprising raw powder or extract or active fraction(s) or compound(s) derived from genus Stevia preferably Stevia rebaudiana leaf to potently enhance the bio-availability and bio-efficacy of a number of supplements or herbal products such as but not limited to Vitamins, minerals, proteins, aminoacids, enzymes Phytosterols, cardioprotectants, carotenoids, joint support ingredients, cartilage protection formulae, fatty acids, Boswellic acids, flavanoids, phenolic compounds, isothiocyanates, energy formulae, weight loss ingredients, immune modulaters, antioxidants, pseudo vitamins like COQ 10 and COQ9, anti inflammatory products, hydration compositions, pre-biotics, pro-biotics, Nutraceuticals and natural herbal products.

The present invention further provides a bioenhancer composition comprising an effective amount of extract or bioactive fraction(s) or compound(s) of Stevia rebaudiana as a bioavailability enhancer optionally along with a known bioenhancer such as piperine, Glycyrrhizin, Niaziridin, cumin extract or pharmaceutically, dietically and nutraceutically acceptable additives/excipients or a carrier.

The present invention still provides a bio-availability and bio-efficacy enhancing agents and compositions comprising compounds naturally present in the leaf, stem, root or whole plant or plant parts of genus Stevia, preferably leaves of Stevia rebaudiana, wherein these compounds may be isolated from the natural source or produced by synthesis or semi-synthesis.

### DESCRIPTION OF THE DRAWINGS

FIG. 1 describes an illustration of one of the embodiments of the invention, a plot drawn between post supplementation blood collection time and the corresponding serum AKBA concentration. Post treatment blood samples were collected at various time points 0.5, 1, 1.5, 2, 2.5, 3, 4 and 5 hrs from the treatment groups.

### DESCRIPTION OF THE INVENTION

The invention will now be described in detail in connection with certain preferred and optional embodiments, so that various aspects thereof may be more fully understood and appreciated.

Improving the bio-availability of a drug candidate is the most important aspect of modern medicine. Especially, the improvement of bio-availability of a drug candidate through the adjunct use of bio-enhancing agent is very important and extremely useful concept. Many natural bio-enhancers have been in use since ancient times. New bio-enhancing agents with potential bio-enhancing activity are in great need to address the poorly bioavailable phytochemicals, nutraceutical and therapeutic agents. During the search for agents to meet this requirement, the inventors have accidentally found that an extract derived from the leaves of Stevia rebaudiana significantly enhanced the bio-availability of 3-O-acetyl-11-keto-beta-boswellic acid, the active component in 5-Loxin® in Sprague Dawley (SD) Rats.

5-Loxin® is a novel Boswellia serrata extract selectively enriched in 30% AKBA (US Patent 2004/0073060A1). It is currently being marketed as an osteoarthritis agent. Its superior efficacy compared to regular Boswellia serrata extract has been well established. The research studies aimed at further enhancing its efficacy have yielded a surprising result. In a selected experiment, SD rats of either sex have randomly assigned to two groups each having six animals. The animals were acclimatized for a week and then fasted over night with free access to water prior to the study. Blood sample (0.2 mL) was collected before administration from Sephanus vein. One group of animals were treated with 100 mg of Boswellia serrata extract standardized to 30% 3-O-acetyl-11-keto-beta-boswellic acid (5-Loxin) and the other group was treated with a composition comprising 100 mg of 5-Loxin and 10 mg of Stevia rubuadiana extract standardized to 33% steviosides.

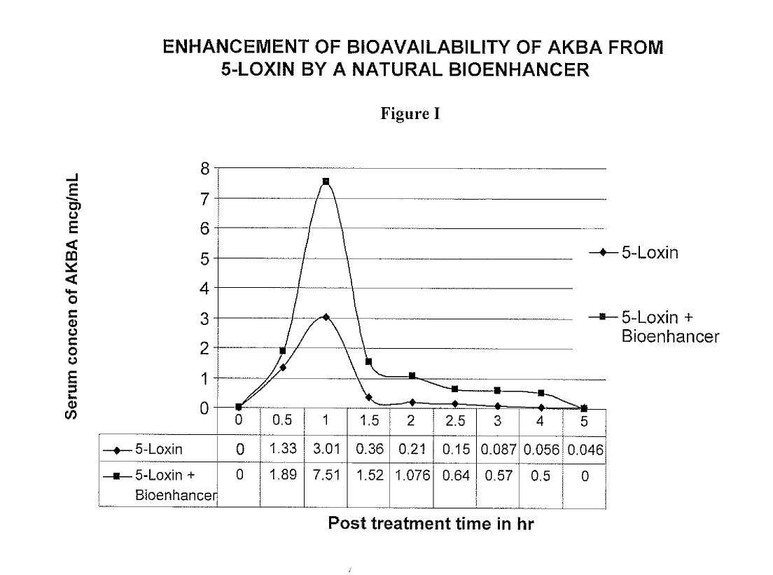

Post treatment blood samples were collected at various time points 0.5, 1, 1.5, 2, 2.5, 3, 4 and 5 hrs from both the treatment groups. The samples were centrifuged at 4° C. for 15 min at 1500×g. The serum samples were de-proteinized and submitted for LC-MS analysis. The serum AKBA content was estimated at each time point and the results are summarized in table 1. A plot drawn between duration and serum AKBA content is depicted in FIG. 1.

Surprisingly, the addition of 10% bio enhancer significantly enhanced serum AKBA concentration as indicated by the increase in AUC (area under the curve) of the curve plotted between serum AKBA concentration versus time after administration. The data clearly shows significant improvement in the bioavailability of 3-O-acetyl-11-keto-β-boswellic acid (AKBA) was observed in the group supplemented with 5-Loxin along with stevia extract compared to the group supplemented with 5-Loxin only. Maximum serum concentration (Cₘₐₓ) was also increased significantly from 3.0 mcg/mL in 5-Loxin treated group to 7.5 meg in bioenhancer 5-Loxin supplemented group. Overall, the bin-availability of AKBA is increased by 90% in the stevia Non-bitter active fraction (NBE) supplemented group compared to the unsupplemented group as shown in Table 1 and diagrammatically represented in FIG. 1.

TABLE 1
SERUM AKBA CONTENT IN mcg/ml

| | | | | GROUP II: 5-LOXIN + |
|---|---|---|---|---|
| | | | GROUP I: 5-LOXIN | BIOENHANCER |
| S. NO. | TIME | | TREATED | TREATED |
| 1 | ½ | HR | 1.33 mcg/ml | 1.89 mcg/ml |
| 2 | 1 | HR | 3.01 mcg/ml | 7.51 mcg/ml |
| 3 | 1½ | HRS | 0.36 mcg/ml | 1.52 mcg/ml |
| 4 | 2 | HRS | 0.21 mcg/ml | 1.076 mcg/ml |
| 5 | 2½ | HRS | 0.15 mcg/ml | 0.64 mcg/ml |
| 6 | 3 | HRS | 0.087 mcg/ml | 0.57 mcg/ml |
| 7 | 4 | HRS | 0.056 mcg/ml | 0.50 mcg/ml |
| 8 | 5 | HRS | 0.046 mcg/ml | NIL |

In one embodiment of the invention, the plant extract of Stevia rebaudiana or its fraction/pure isolates used are derived from any plant parts of Stevia rebaudiana like leaves, stem, root, whole plant and in particular the leaves.

One more embodiment of the invention related to administration of the bioenhancing composition. The composition is administered through oral, parenteral, nasal, inhalation including nebulisers, rectal, vaginal, transdermal, occular and any other suitable routes.

In yet another embodiment, the bioenhancing effect of the extracts/fractions/pure isolates of Stevia rebaudiana either alone or in combination with piperine is selective in enhancing the bioavailability/bioefficacy of a drug, nutraceutical, and herbal drug/formulation.

The percentage of each of the stevia compound/component in the bioenhancing composition varies from 0.01-80%. The total percentage of the stevia components in the bioenhancer composition varies in the range of 0.01-80%.

In another embodiment, the concentration of the bioehancer derived from Stevia rebaudiana in the composition comprising bioenhancer and one of herbal extracts/pharmaceutical drugs/phytochemical compounds and other agents, whose bioavailability has to be enhanced is in the range of 0.01 to 40%.

In yet another embodiment, the dosage of bioenhancer from Stevia rebaudiana as extract is in the range of 0.01 to 50 mg/kg/body weight and piperine is in the range of 0.01 to 12 mg/kg/body weight.

Another embodiment, the dosage of bioenhancer from Stevia rebaudiana as bioactive fraction or pure compound is in the range of 0.01 to 40 mg/kg/body weight, preferably 30 mg/kg/body weight, and piperine is in the range of 0.01 to 10 mg/kg/body weight, preferably 8 mg/kg/body weight.

The present invention provides bio-efficacy and bio-availability facilitating composition comprising, raw powder or extract or active fraction(s) or compound(s) derived from the whole plant or plant part(s) of genus Stevia preferably Stevia rebaudiana leaf for enhancing the bio-availability of therapeutic agents, cosmetic agents, and active ingredients in supplements, food ingredients and beverages selected from but not limited to drugs, nutrients (micro and macro nutrients) vitamins, minerals, proteins, amino acids, enzymes, nutraceuticals, herbal drugs/products and antioxidants.

Accordingly, bio-efficacy and bio-availability facilitating composition of the present invention comprises of,
i. Bioenhancer selected from leaf powder, extract, active fraction or fractions, compound or compounds derived from Stevia rebaudiana leaves as an active ingredient either alone or in combination;
ii. one or more active ingredient selected from but not limited to drugs, nutrients, vitamins, nutraceuticals, herbal drugs/products, phytochemicals, micronutrients, antioxidants.
iii. optionally comprising one or more pharmaceutically, dietically and nutraceutically acceptable additives/excipients and
iv. further optionally containing an effective amount of one or more of known bio-availability enhancers selected from but not limited to extracts or fractions of Piper nigrum or Piper longum or Glycyrrhiza glabra or Zingiber officinale or Piperine, Glycyrrhizin, Niaziridin, Cumin extracts.

The agents and/or compositions of the present invention can be administered in a therapeutically effective amount for use in humans or animals or subjects in need thereof.

An ‘Agent’ quoted in the present context refers to an extract or fraction or pure compound derived from Stevia, preferably Stevia rebaudiana.

In yet another embodiment, the dosage of bioenhancer from Stevia rebaudiana, wherein the amount of raw powder or extract or active fraction(s) or compound(s) derived from genus Stevia preferably Stevia rebaudiana leaf used is in the range of about 0.1 to 250 mg per a dose of the pharmaceutical drug or herbal extract or nutraceutical compound.

In yet another embodiment, the dosage of bioenhancer from Stevia rebaudiana, wherein the amount of Stevia rebaudiana fractions/pure isolates used is in the range of about 0.1 to 75 mg irrespective of the amount of drug in the composition preferably in the range of 1 to 30 mg per a dose of the pharmaceutical drug or herbal extract or nutraceutical compound irrespective of the amount of drugs in the composition.

In other embodiment of the invention, the bio-availability and bio-efficacy enhancing composition comprises compounds or components naturally exists in Stevia rebaudiana, wherein the compounds may be obtained by isolation from a plant source or prepared by synthesis or semi-synthesis. These stevia natural compounds include but not limited to Beta Amyrin acetate, Anethole, Apigenin-4′-o-beta-d-glucoside, Austroinulin, 6-o-acetyl Austroinulin, Austroinulin-6-acetate, Austroinulin-7-acetate, Avicularin, Benzyl alcohol, alpha-Bergamotene, Bisabolene, Borneol, beta-Bourbonene, delta-Cadinene, gamma-Cadinene, alpha-Cadinol, delta-Cadinol, T-Cadinol, Caffeic acid, alpha-Calacorene, Campesterol, Camphor, beta-Carotene, Carvacrol, Caryophyllene, Caryophyllene oxide, beta-Caryophyllene, Centaureidin, Chlorogenic acid, Chlorophyll A, Chlorophyll B, 1-8 Cineol, Citric acid, alpha-Copaene, Cosmosiin, alpha-Cubebene, beta-Cubebene, Cumin aldehyde, Cymene, para, Cynaroside, Daucosterol, Diterpene glycosides, Dulcoside A, Dulcoside B, Elemen, beta, Elemene, gamma, Essential oil (Stevia rebaudiana), Eugenol, Eugenol methyl ether, Farnesene, beta trans, Fluoride, Foeniculin, Formic acid, Fura, 2-pentyl, Geraniol, Germacrene D, Gibberellic acid, Gibberellin A-20, Glucose, Glucosilsteviol, Hex-3-en-1-ol-2-methyl-butanoate, Hexan-1-al, alpha-Humulene, Indole-3-acetonitrile, beta-Ionone, beta epoxy-Ionone, Jhanol, Kaempferol-3-o-rhamnoside, (−) Kaur-16-en-19-oic acid, (−) Kaurene, Limonene, Linalool, Linalool, cis oxide, Linalool, trans oxide, Lupeol, Lupeol palmitate, Luteolin-7-O-glucoside, Myrcene, Myrtenal, Myrtenol Nerolidol, Oct-1-en-3-ol, Octa-2-3-dione, Octan-3-ol, Pentanoic acid, Phenylethyl valerate, alpha-Pinene, beta-Pinene, Pinocarveol, Pinocarvone, Polystachoside, Protein, Quercetin, Quercitrin, iso-Quercitrin, Rebaudioside A, Rebaudioside A cont., Rebaudioside B, Rebaudioside C, Rebaudioside D, Rebaudioside E, Rebaudioside F, Rutin, Sabinene, Scopoletin, beta-Selinene, beta-Sitosterol, Spathulenol, Sterebin A, Sterebin B, Sterebin C, Sterebin D, Sterebin E, Sterebin F, Sterebin G, Sterebin H, Stevia rebaudiana compound 1a-3, Stevia rebaudiana compound a-1, Stevia rebaudiana compound a −3, Stevia rebaudiana compound a-4, Stevia rebaudiana compound a-6, Stevia rebaudiana compound a-d, Stevia rebaudiana compound x, Stevia rebaudiana glycoside, Stevia rebaudiana glycoside a-3, Stevia rebaudiana sweet principle m, Steviol, iso-Steviol, Steviolbioside, Steviolmonoside, Stevioside, Stevioside cont., Stevioside a-3, Stevioside sweetener, Stigmasterol, Stigmasterol-3-O-beta-d-glucoside, Sucrose, Tannin (Stevia rebaudiana), Tartaric acid, Terpinen-4-ol, gamma-Terpinene, alpha-Terpineol, Terpinolene, Umbelliferone, trans-Verbenol, and Xanthophyll.

One embodiment of the invention provides a stevia bio-availability composition in which the active ingredient for bio-availability enhancement are selected from but not limited to the group consisting of antibiotics, antiobese products, antidiabetics, antifungals, antivirals, anticancer, cardiovascular products, anti-inflammatory, antiarthritic agents, joint support ingredients, antiTB/antileprosy products, antihistaminic/respiratory drugs, immunity modulators, cholesterol lowering agents, C-reactive protein inhibitors, antiulcer drugs and phytochemicals.

The further embodiment of the present invention relates to providing of raw powder or extract or active fraction(s) or compound(s) derived from genus Stevia preferably Stevia rebaudiana leaf to potently enhance the bio-availability and bio-efficacy of a number of supplements or herbal products such as but not limited to vitamins, minerals, proteins, aminoacids, enzymes, phytosterols, cardioprotectants, carotenoids, joint support ingredients, cartilage protection formulae, fatty acids, flavanoids, phenolic compounds, isothiocyanates, energy formulae, weight loss ingredients, immune modulators, antioxidants, pseudo vitamins like COQ10 and COQ9, anti inflammatory, hydration compositions, pre-biotics and pro-biotics.

In yet another embodiment the herbal product/drug may be selected from but not limited to Boswellia serrata, Echinaceae, Tinospora cordifolia, Picrorrhiza kurroa, Aegle marmelos, Andrographis paniculata, Emblica ribes, Asparagus racemosus, Terminalia chebula, Withania somnifera, Centella asiatica, Garcinia mangostana, Curcuma longa, Murraya koenigii, Moringa oleifera extracts, enriched fractions or pure compounds and/or their mixture thereof.

In a further embodiment of the invention the Stevia bioenhancer as described herein can be used to improve the bio-enhancement and/or bio-efficacy of the active compounds or phytochemicals selected from but not limited to Boswellic acids, xanthones, withanolides, flavanoids, lignans, stilbene compounds, curcuminoids, vascicine/vascinone, ellagic acid, Furano diterpenes, Irridoid glycosides, Embilin, Saponins, Tannins, Triterpenes, Triterpene saponins, Carbazole alkaloids and flavonoid glycosides and/or their mixture thereof.

The Boswellia serrata components can be selected from the extract(s), enriched extracts, fraction(s), enriched fractions, pure compound(s) or mixtures thereof.

The AKBA concentration in the extract(s), enriched extracts, fraction(s), enriched fractions can vary from 0.1% to 99%, preferably 1% to 60% and more preferably 3% to 45%.

Yet another embodiment, the antibiotic used can be selected from but not limited to the group consisting of quinolones, macrolides, cephalosporins, penicillins and aminoglycosides wherein quinolone is selected from but not limited to the group consisting of Ciprofloxacin, Pefloxacin, Ofloxacin and Norfloxacin; the macrolide is selected from but not limited to the group consisting of Erythromycin, Roxythromycin and Azithromycin; the cephalosporins is selected from but not limited to the group consisting of Cefalexin, cefatrioxone, cefixime and Cefadroxil; the penicillin is selected from but not limited to the group consisting of Amoxycillin and Cloxacillin; and aminoglycocide is selected from but not limited to the group consisting of Amikacin and Kanamycin.

In yet another embodiment, the anti-fungal drug may be selected from but not limited to the group consisting of Fluconazole, Amphoterincin B and Ketoconazole.

In yet another embodiment, the anti-viral drug may be selected from but not limited to the group consisting of Acyclovir and Zidovudine.

In yet another embodiment, the anti-cancer drug may be selected from but not limited to the group consisting of Methotrexate, 5-Fluorouracil, Doxorubicin, Taxol and Cisplatin.

In yet another embodiment, the Cardiovascular drug may be selected from but not limited to the group consisting of Amlodipin, Lisinopril, propranolol, and Atenolol.

In yet another embodiment, the CNS drugs may be selected from but not limited to the group consisting of Alprazolam, Heloperidol.

In yet another embodiment, the anti-inflammatory/anti-arthritic drug may be selected from but not limited to the group consisting of Diclofenac, Piroxicam, Nimesulide and Rofecoxib.

In yet another embodiment, the anti-TB/anti-Leprosy drug may be selected from but not limited to the group consisting of Rifampicin, Ethionamide, Ethambutol and Dapsone.

In yet another embodiment, the anti-histamine/drugs for respiratory disorders may be selected from but not limited to the group Salbutamol, Theophylline, Bromhexine and Loratidine.

In yet another embodiment, the corticosteroids may be selected from but not limited to the group Prednisolone, dexamethasone and Betamethasone.

In yet another embodiment, the immunosuppressant may be selected from but not limited to the group Cyclosporin A, Tacrolimus, Mycophenolatemofetil.

In yet another embodiment, the anti-ulcer compound may be selected from but not limited to the group Ran itidine, Cimetidine and Omerprazole.

In yet another embodiment, the nutrients may be selected from but not limited to sugar, carbohydrate, fats and proteins.

In yet another embodiment, the vitamins may be selected from but not limited to the group Vitamin A, Vitamin B1, Vitamin B6, Vitamin B12, Vitamin C and Folic acid.

In yet another embodiment, the anti-oxidant may be selected from but not limited to the group β-carotene, Silymarin, Selenium, Lycopene and Ellagiogallotannins.

In another embodiment, the natural herbal products may be selected from but not limited to Curcumin, Boswellic acid and Rutins.

In yet another embodiment, the essential micronutrients may be selected from but not limited to Methionine, Lysine, Leucine, Valine, Isoleucine, Zinc, Calcium, Glucose, Potassium, Copper and Iron.

As applicable to any mechanism of action the products of this invention contribute in a synergistic and/or additive manner so that most drugs and nutraceuticals in presence of the products described in the present art are more bioavailable or bioefficaceous as a result of one or more of the mechanisms. The bioavailability and the bioefficacy of drugs and nutraceuticals are also relevant to animal health besides being important for humans. The invention therefore is also intended to be used in veterinary preparations.

In other important embodiment of the invention, the bio-availability and bio-efficacy enhancing composition comprises compounds naturally exists in Stevia rebaudiana, wherein the process for preparation of extract(s)/fraction(s) of plants involves the use of water, alcohol, combinations of water and alcohol, halogenated hydrocarbons, ketones, ethers as solvents.

Specific dosage form for bio-availability enhancement includes, for example, oral agents such as tablets, soft capsule, hard capsule, pills, granules, powders, emulsions, suspensions, syrups, inhalation aids, aerosol, transdermal delivery systems and other drug delivery systems and pellets; and parenteral agents such as injections, drops, suppositories and the like.

The amount of composition that will be effective in the treatment of a particular disorder or condition will depend on the nature of the disorder of the condition, which can be determined by standard clinical techniques. In addition, the in vitro and in vivo assays may optionally be employed to help identify optimal dosage ranges. The precise dose to be employed in the formulation will depend on the route of administration, and the seriousness or advancement of the diseased condition, and should be decided according to the practitioner and each patient's circumstances. Effective dosages may be extrapolated from dose response curves derived from in vitro or animal model test systems. For example an effective amount of the composition of the invention is readily determined by administering graded doses of the composition and observing the desired effect.

In a further embodiment of the invention, the compositions comprising of raw powder or extract or active fraction(s) or compound(s) derived from genus Stevia preferably Stevia rebaudiana leaf or their compositions for bio-enhancement are formulated into any food and drink forms such as solid food like chocolate or nutritional bars, semisolid food like cream or jam, or gel. Contemplation was also made to formulate the product of the invention into a beverage and the like, such as refreshing beverage, coffee, tea, milk-contained beverage, lactic acid bacteria beverage, drop, candy, chewing gum, chocolate, gummy candy, yoghurt, ice cream, pudding, soft adzuki-bean jelly, jelly, cookie and the like.

In yet another embodiment, the invention further relates to the method of using the bioenhancing composition in humans or animals in need thereof, wherein the method comprises supplementing the said humans or animals with an effective amount of a composition comprising:
i) Bioenhancer is selected from raw powder or extract or active fraction(s) or compound(s) derived from genus Stevia preferably Stevia rebaudiana leaf as an active ingredient in combination with;
ii) One or more pharmaceutical active ingredients/drugs or herbal product or Nutraceutical compounds or phytochemicals;
iii) Optionally one or more pharmaceutically, dietically and nutraceutically acceptable additives/excipient and
iv) Further optionally an effective amount of one or more other known bioenhancers selected from but not limited to extract/fraction of Piper nigrum or Piper longum or Glycyrrhiza glabra or Zingiber officinale or Piperine or Glycyrrhizin or Niaziridin or Cumin extracts.

One more embodiment of the present invention provides a process for producing active extracts and active fractions having bio-availability enhancing effect.

The following examples, which include preferred embodiments, will serve to illustrate the practice of this invention, it being understood that the particulars shown are by way of example and for purpose of illustrative discussion of preferred embodiments of the invention.

### EXAMPLES

### Example 1

Process for producing stevia extract: Stevia rebaudiana leaf raw material (1 kg) was soaked in 50% aqueous methanol (4 L) and extracted at 80° C. After 2 h, the extract was separated through filtration and the spent raw material was re-extracted with 50% aqueous methanol (4×3 L) at 70-80° C. The extracts were combined and concentrated to 10% total solids. The pH of the solution was then adjusted to 11 using Ca(OH)₂. The precipitate was filtered through a filter paper, and washed with 2 L water and the filtrate and the washings were combined and neutralized back to pH 7 using citric acid. The precipitate formed was filtered again and the filtrate was evaporated under vacuum to obtain a solid (280 g). The solid has shown 5% of Rebaudioside A; 24% of Stevioside; 3% of Rebaudioside C and 1% of Dulcoside A by HPLC analysis

### Example 2

Process for preparing the non-bitter active fractions (NBE) from Stevia rebaudiana: Stevia rebaudiana leaf raw material (1 kg) was soaked in ethyl acetate (4 L) and subjected to gentle reflux at 75-80° C. After 2 h, the extract was separated through filtration, washed with ethyl acetate (0.6 L) and the spent raw material was dried by open drying. The dried spent raw material was then extracted with water (3 L) at 70-80° C. The extract was filtered and the raw material was re-extracted with water (2 L). The aqueous extracts were combined and concentrated to half the volume. The pH of the solution was then adjusted to 11 using CaO or Ca(OH)₂. The precipitate was filtered through a filter paper, and washed with 2 L water and the filtrate and the washing were combined and neutralized back to pH 7 using citric acid. The precipitate formed was filtered again and the filtrate was evaporated under vacuum to obtain a concentrate with 30% total solids (TS) and the solid was separated again and the solution was spray dried to obtain non-bitter stevia extract (stevia NBE) as a solid (229 g). The solid has shown 3% of Rebaudioside A; 13% of Stevioside; 2% of Rebaudioside C and 1% of Dulcoside A by HPLC analysis.

### Example 3

The non bitter stevia extract (NBE) (5 g) was dissolved in water (15 mL) and loaded to R 20 resin column (50 mL, synthetic adsorbent) and the column was washed with water (100 mL). Eluted with 90% Methanol and evaporated to obtain glycosides enriched stevia fraction. The enriched fraction showed 19% of Rebaudioside A; 35% of Stevioside; 7% of Rebaudioside C and 3% of Dulcoside A by HPLC analysis.

The above extracts are further purified using column chromatography on silica column using chloroform-methanol mixtures to obtain the pure compounds Rebaudioside A; Stevioside; Rebaudioside C and Dulcoside A.

### Example 4

Bio-availability/bio-efficacy facilitating action of Stevia extract on 5-Loxin: Eight healthy Sprague Dawley (SD) rats were selected, acclimatized for 7 days and allocated randomly into two treatment groups, each containing 4 animals. All the study animals were fasted overnight at free access to water. Blood sample (0.2 mL) was collected from Sephanus vein before the administration of study product. SD rats were orally treated with either 100 mg 5-Loxin alone or 100 mg 5-Loxin+10 mg bio-enhancer stevia NBE obtained as described in example 2 suspended in 0.5% CMC.

Post treatment blood samples were collected at various time points 0.5, 1, 1.5, 2, 2.5, 3, 4 and 5 hrs from both the treatment groups. The samples were centrifuged at 4° C. for 15 min at 1500×g. The serum samples were de-proteinized and submitted for LC-MS analysis. The serum AKBA content was estimated at each time point and the results are summarized in table 1. A plot drawn between duration and serum AKBA content is depicted in FIG. 1.

### Example 5

### Method of Estimating the Enhancement of Bioavailability of AKBA in 5-Loxin

The bioavailability enhancement of AKBA present in 5-Loxin was estimated by using LC-MS.

### Standard Preparation for Analysis:

Weighed about 5.0 mg of standard into 50 ml volumetric flask and dissolved in methanol and make up to the mark and filtered through 0.45 μm membrane filter.

### Sample Preparation for Analysis:

Take 500 μL of serum and 500 μL of 20% Trichloro acetic acid (TCA) in 10 ml volumetric flask and make up to the mark with methanol and filtered through 0.45 μm membrane filter.

### Preparation of Rat Serum:

SD rats were anaesthetized by using anesthetic ether and blood was collected from sinus using a glass capillary. Allowed it to clot and collected blood was centrifuged (at room temperature) at 3000 rpm and supernatant liquid was collected.

### HPLC Instrumentation:

The HPLC system, Supplied by M/s Shimadzu comprising LC-10AT VP pumps, SCL-10A VP auto injector and Phenomenex, Luna, Phenyl-Hexyl, 5μ, (250×4.6 mm) column was used at 30° C. temperature. Isocratic elution was carried out with Methanol: 0.1% (v/v) acetic acid in water (90:10, v/v) at a flow rate of 1 ml/min, detection was at 248 nm using SPD-M10 AVP photodiode array detector. Class VP software was used for integration and calibration. Evaluation was via peak areas with linear regression.

### LC-MS Instrumentation:

Liquid chromatography was performed on Phenomenex, Luna, Phenyl-Hexyl, 5μ, (250×4.6 mm) column, using Agilent 1100 series binary pump with Chem station controller. The mobile phase consists of Methanol: 0.1% (v/v) acetic acid in water (90:10, v/v) at a flow rate of 1 ml/min. The liquid chromatographic system was couple to Agilent 1100 series LC-MSD equipped with electrospray ionization with the following optimized instrumental settings, fragmentor voltage 160 Volts; capillary voltage 4000 V, nebulizer pressure 50 psi, dry gas flow 10 L/min and dry gas temperature is 350° C. The response of 5-loxin was measured using mass detector tune to Positive ion mode with m/z 471.4 in selective ion monitoring (SIM) method. Agilent Chemstation data software was used for data integration.

### LC-MS Analysis:

The samples were injected (20 μL) and the analysis was carried out with optimized instrument settings, integrate all the chromatograms with Agilent Chemstation data software. The total time per analysis for each elute was about 10.9 min. The molecular ion was detected and quantitated at m/z 513.5.

$$\% of 5 \text{-} Loxin = \frac{Area of sample \times std . cone \times std . purity }{Area of standard \times sample cone} .$$

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1-20. (canceled)

21. A bioenhancing composition comprising:
a material derived from a plant of the genus Stevia, said material being in the form of a raw powder, an extract, at least one active fraction, at least one compound, or a mixture thereof as a bioenhancer,
wherein said material is present in an amount effective to enhance the bio-efficacy or bio-availability of at least one active compound selected from the group consisting of pharmaceutical drugs, herbal products, nutraceutical compounds, phytochemicals, and mixtures thereof.

22. A composition according to claim 21, further comprising a therapeutically effective amount of said at least one active compound selected from the group consisting of pharmaceutical drugs, herbal products, nutraceutical compounds, phytochemicals, and mixtures thereof.

23. A composition according to claim 21, wherein said material derived from a plant of the genus Stevia is derived from Stevia rebaudiana.

24. A composition according to claim 22, wherein said material derived from a plant of the genus Stevia is derived from Stevia rebaudiana.

25. A composition according to claim 21, wherein the at least one active compound is selected from the group consisting of antibiotic agents, antiobesity agents, antidiabetic agents, antifungal agents, antiviral agents, anticancer agents, cardiovascular agents, anti-inflammatory agents, anti-arthritic agents, antihistamines, respiratory agents, immune modulators, cholesterol lowering drugs, C-reactive protein inhibitors, antiulcer drugs, antimicrobial agents, antitubercular agents, antileprosy agents, anti-inflammatory agents, anti-arthritic agents, antihistaminics, CNS drugs, respiratory distress relieving drugs, immunosuppressants, herbal formulations, and mixtures thereof.

26. A composition according to claim 22, wherein the material derived from a plant of the genus Stevia is used in the range of about 0.1 to 250 mg.

27. A composition according to claim 26, wherein the material derived from a plant of the genus Stevia is derived from Stevia rebaudiana leaf.

28. A composition according to claim 22, wherein the material derived from a plant of the genus Stevia is used in the range of about 0.1 to 75 mg.

29. A composition according to claim 22, wherein the material derived from a plant of the genus Stevia is used in the range of 1 to 30 mg.

30. A composition according to claim 21, wherein bioenhancing agent may be used optionally in combination one or more other bioenhancing agents selected from piperine, glycyrrhizin, niaziridin, cumin extract and further optionally in combination with an additive or a carrier.

31. A composition according to claim 21, wherein the active compound is a herbal product selected from the group consisting of Boswellia serrata, Garcinia mangostana, Curcuma longa, Echinaceae, Tinospora cordifolia, Picrorrhiza kurroa, Aegle marmelos, Andrographis paniculata, Emblica ribes, Asparagus racemosus, Terminalia chebula, Withania somnifera, Centella asiatica, Murraya koenigii, Moringa oleifera, extracts thereof, enriched fractions thereof, pure compounds isolated therefrom, or mixtures thereof.

32. A composition according to claim 31, wherein the active compound is a herbal product derived from Boswellia serrata, said herbal product derived from Boswellia serrata being selected from the group consisting of at least one extract, at least one enriched extract, at least one fraction, at least one enriched fraction, at least one pure compound, or mixtures thereof.

33. The composition according to claim 32, wherein the active compound is a herbal product derived from Boswellia serrata, said herbal product comprising 3-O-acetyl-11-keto-beta-boswellic acid (AKBA).

34. A composition according to claim 33, wherein the 3-O-acetyl-11-keto-beta-boswellic acid (AKBA) concentration in the herbal product derived from Boswellia serrata is 0.1% to 99%.

35. A composition according to claim 33, wherein the 3-O-acetyl-11-keto-beta-boswellic acid (AKBA) concentration in the herbal product derived from Boswellia serrata is 1% to 60%.

36. A composition according to claim 33, wherein the 3-O-acetyl-11-keto-beta-boswellic acid (AKBA) concentration in the herbal product derived from Boswellia serrata is 3% to 45%.

37. The Boswellia serrata extract according to claim 33, wherein the extract is an enriched extract standardized to 30% 3-O-acetyl-11-keto-beta-boswellic acid (AKBA).

38. A composition according to claim 21, wherein the active compound is selected from to the group consisting of Boswellic acids, xanthones, withanolides, flavanoids, lignans, stilbene compounds, curcuminoids, vascicine, vascinone, ellagic acid, rutins, Furano diterpenes, Irridoid glycosides, Embilin, Saponins, Tannins, Triterpenes, Triterpene saponins, Carbazole alkaloids, flavonoid glycosides, and mixtures thereof.

39. A composition according to claim 21, wherein the material derived from a plant of the genus Stevia is derived from a leaf, stem, root, whole plant or leaves of Stevia rebaudiana.

40. A composition according to claim 22, wherein the composition comprising the bioenhancer material derived from a plant of the genus Stevia and the active compound is administered through oral, parenteral, nasal, inhalation, including nebulizers, rectal, vaginal, transdermal, or occular routes.

41. A composition according to claim 21, wherein the material derived from a plant of the genus Stevia is used alone or in combination with Piperine in enhancing the bioavailability or bioefficacy of the at least one active compound.

42. A composition according to claim 22, wherein the percentage of material derived from a plant of the genus Stevia varies from 0.01-80%.

43. A composition according to claim 21, wherein the total percentage of material derived from a plant of the genus Stevia varies in the range of 0.01-80%.

44. A composition according to claim 22, wherein the concentration of the bioenhancer material derived from a plant of the genus Stevia is in the range of 0.01 to 40%;
said bioenhancer material being derived from Stevia rebaudiana.

45. A composition according to claim 41, wherein:
the bioenhancer material derived from a plant of the genus Stevia is derived from Stevia rebaudiana, and is used in a dosage in the range of 0.01-50 mg/kg/body weight; and
Piperine is used in a dosage in the range of 0.01 to 12 mg/kg/body weight.

46. The composition as claimed in claim 45, wherein:
the bioenhancer material is used in a dosage in the range of 0.01-40 mg/kg/body weight; and
Piperine is used in a dosage in the range of 0.01-10 mg/kg/body weight.

47. The composition as claimed in claim 45, wherein:
the bioenhancer material is used in a dosage in the range of 30 mg/kg/body weight; and
Piperine is used in a dosage in the range of 8 mg/kg/body weight.

48. The method of enhancing bio-efficacy or bio-availability of at least one active compound in humans or animals in need thereof, wherein the method comprises administering to said humans or animals an effective amount of a composition comprising:
i) a bioenhancer in an amount effective to enhance bio-efficacy or bio-availability of said at least one active compound, said bioenhancer being a material derived from a plant of the genus Stevia, said material being in the form of a raw powder, an extract, at least one active fraction, at least one compound, or a mixture thereof;
ii) said at least one active compound, said at least one active compound being selected from the group consisting of pharmaceutical drugs, herbal products, nutraceutical compounds, phytochemicals, and mixtures thereof; and
iii) optionally one or more pharmaceutically, dietically and nutraceutically acceptable additives or excipients; and
iv) optionally an effective amount of one or more other known bioenhancers selected from the group consisting of Piperine; Glycyrrhizin; Niaziridin; an extract or fraction of Piper nigrum, an extract or fraction of Piper longum; an extract or fraction of Glycyrrhiza glabra; an extract or fraction of Zingiber officinale; an extract or fraction of Cumin; or a mixture thereof.

49. The method of claim 48, wherein said material derived from a plant of the genus Stevia is derived from Stevia rebaudiana leaves.

50. A composition according to claim 33, wherein said therapeutically effective amount of said at least one active compound is a therapeutically effective amount of a herbal product comprising 3-O-acetyl-11-keto-beta-boswellic acid (AKBA) derived from Boswellia serrata; and wherein said at least one active compound and said material derived from a plant of the genus Stevia are present in a ratio of 10:1 by weight.
